(19) United States
(12) Patent Application Publication
(10) Pub. No.: US 2018/0200480 A1
(43) Pub. Date: Jul. 19, 2018

(54) CATHETER EXTENSION DEVICE

(71) Applicant: Turnstone Technologies, LLC, Carson City, NV (US)

(72) Inventors: Aaron Chiu, El Paso, TX (US); Enrique Delgado Macias, Ciudad Juarez (MX)

(21) Appl. No.: 15/872,930

(22) Filed: Jan. 16, 2018

Related U.S. Application Data
(60) Provisional application No. 62/446,361, filed on Jan. 13, 2017.

(51) Int. Cl. A61M 25/00; A61F 2/958; A61L 29/14; A61L 29/02; A61L 29/06; A61L 29/08; A61L 29/16

(52) U.S. Cl. CPC A61M 25/0074; A61F 2/958; A61M 25/0097; A61M 25/0045; A61M 25/005; A61F 2002/9517; A61L 29/02; A61L 29/06; A61L 29/085; A61L 29/16; A61L 29/14

(57) ABSTRACT

The present invention includes a catheter extension device, and method of use, for coaxially extending access of a catheter into selective targets of the vasculature and delivering therapeutics.

This application relates to, and claims the benefit of the filing date of, co-pending U.S. Provisional Patent Application Ser. No. 62/446,361, entitled CATHETER EXTENSION DEVICE, filed on Jan. 13, 2017, the entire contents which are incorporated herein by reference for all purposes.

### BACKGROUND OF THE INVENTION

### Field of the Invention

The present invention relates to catheter devices, and more particularly to an apparatus and a method for accessing a selective or targeted area in the vasculature for delivery of therapeutics.

### Description of the Related Art

Engaging a catheter in the arterial vessel can be challenging when seating the catheter in the ostium of the selected artery. When engaging a catheter for vascular intervention, lining proper backup support may be necessary for optimal delivery of a therapeutic device. Back up support is the ability of the catheter to remain in a stable position and the ability for the user to provide a stable platform during the advancement of the therapeutic device through the catheter. Often, back up support may not be sufficient when the catheter is pushed distally against resistance. When back up support fails, the catheter can be forced out of the ostium and its originally seated position. This failure in back up support can lead to delays and complications in vascular intervention procedures. The present invention provides a catheter extension device that coaxially stabilizes the catheter regardless of whether the catheter being used has the proper back up support. The present invention enables stable deep engagement of a catheter and the delivery of therapeutics that require significant stability to reach selected targets.

### SUMMARY

Provided is a catheter extension device, and method of use, for accessing a selective or targeted area in the vasculature for delivery of therapeutic devices.

### BRIEF DESCRIPTION OF THE DRAWINGS

For a more complete understanding of the features and advantages thereof, reference is now made to the following Detailed Description taken in conjunction with the accompanying drawings, in which:

FIG. 1 illustrates an embodiment of a catheter extension device;

FIG. 2 illustrates various embodiments of a channel reinforcement;

FIG. 3 illustrates a collar formed with rounded edges;

FIGS. 4A-4C illustrate a therapeutic device being inserted into the catheter extension device;

FIG. 5 illustrates the contact point between the collar and a distal extension segment;

FIG. 6 illustrates the catheter extension device in preparation for delivery of a stent;

FIG. 7 illustrates the catheter extension device being used for coronary intervention;

FIG. 8 illustrates the catheter extension device being used for peripheral intervention; and

FIG. 9 illustrates the catheter extension device being used for neurovascular intervention.

### DETAILED DESCRIPTION OF THE INVENTION

In the following discussion, numerous specific details are set forth to provide a thorough understanding of the present invention. However, those skilled in the art will appreciate that the present invention may be practiced without such specific details. In other instances, certain specific details, and the like have been omitted inasmuch as such details are not considered necessary to obtain a complete understanding of the present invention, and are considered to be within the understanding of persons of ordinary skill in the relevant art.

The present invention includes a catheter extension device for accessing a selective or targeted area in the vasculature. The catheter extension device may be used by a user to assist in the delivery of a therapeutic device. In an embodiment, the catheter extension device may be used in conjunction with standard or specialized guiding catheters or guiding sheaths in interventional radiology procedures. The catheter extension device may be coaxially passed through the guiding catheter or guiding sheath to extend the user's reach to a target area in the vasculature. When used, the catheter extension device may extend beyond the distal end of the guiding catheter or guiding sheath positioned at an arterial ostium to enable more selective access of target.

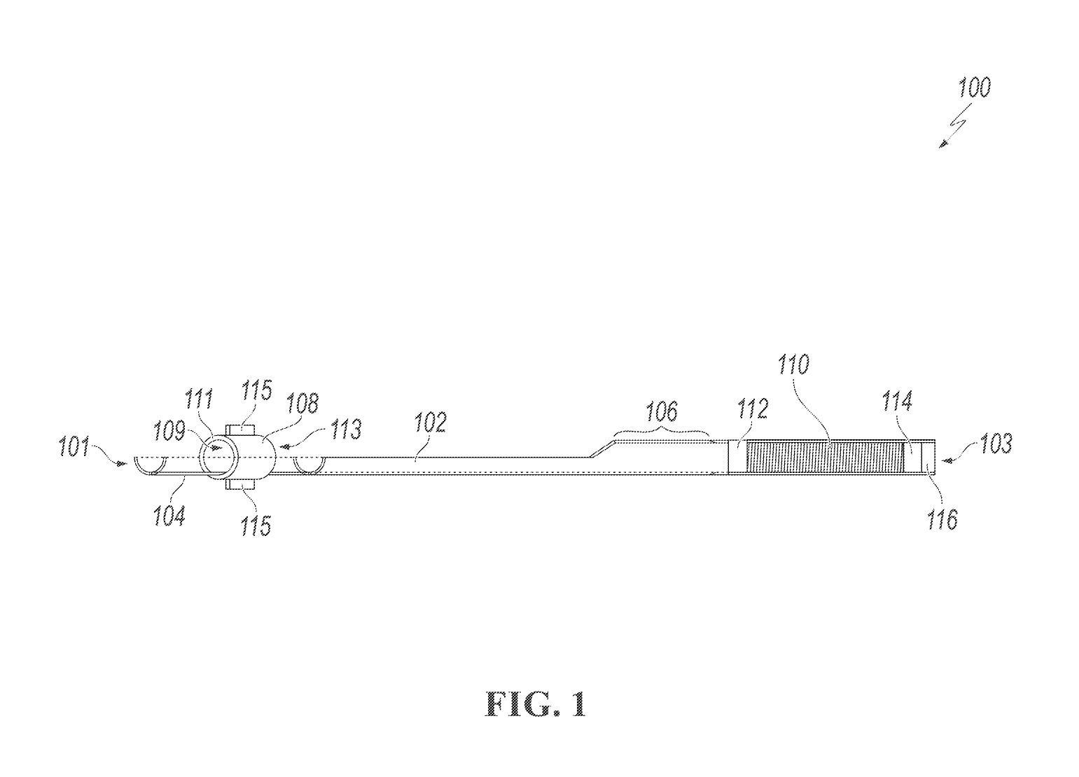

Turning to FIG. 1, in an embodiment, the catheter extension device 100 may comprise a profiled channel 102, a channel reinforcement 104, a collar 106, a trackable hub 108, and a distal extension segment 110. The catheter extension device 100 may comprise a proximal end 101 and a distal end 103 with an end of the profiled channel 102 at the proximal end 101 and an end of the distal extension segment 110 at the distal end 103. The collar 104 may be positioned at a transition area of the catheter extension device 100 between the profiled channel 102 and the distal extension segment 110. The profiled channel 102 may extend from the proximal end 101 of the extension device 100 to a first end of the collar 106. The distal extension segment 110 may extend from a second end of the collar 106 to the distal end 103 of the extension device 100.

The trackable hub 108 may be formed substantially in the shape of a hollowed cylinder comprising an opening 109 extending completely through the hub 108 from a first end 111 to a second end 113. The catheter extension device 100 may be fitted coaxially through the trackable 108 by inserting the profiled channel 102 through the opening 109 in the trackable hub 108. The opening 109 in the hub 108 may be sized such that the hub 108 may be moved along the catheter extension device 100. The trackable hub 108 may be tracked forwards and backwards along the profiled channel 102 enabling the catheter extension device 100 to be torqued and tracked within the guiding catheter or sheath. The trackable hub 108 may be formed with one or more fins 115 extending outwards away from the body of the trackable hub 108. The one or more fins 115 may be formed in the shape of a triangle, square, rectangle, and the like. In an embodiment, the trackable hub 108 may comprise two fins 115 with each fin 115 positioned on opposite sides of the hub 108 with each fin 115 extending from the hub 108 in opposite directions from one another. The one or more fins 115 may allow for the user to manipulate the trackable hub 108 to track the catheter extension device 100.

The catheter extension device 100 may further comprise a proximal marker band 112, a distal marker band 114, and a distal tip 116. Each of the proximal and distal marker bands 112, 114 may be made of materials including but not limited to platinum-iridium, tantalum, polymer with radiopaque fillers, and the like. The distal tip 116 may be formed with atraumatic and or soft properties to reduce the risk of puncture or damaging vessel walls when tracking the catheter extension device through arterial vessels.

The profiled channel 102 may extend from the proximal end 101 of the extension device 100 towards the collar 106. The profiled channel 102 may be formed in the shape of a half round channel with a semi-cylindrical concave groove 117 cutout made through a half cylinder. The groove 117 cutout may be made such that the profiled channel 102 may be substantially formed in shape of a half round channel similar to a “U” with the groove 117 cutout open upwards. The profiled channel 102 may be formed such that the remaining rounded outer surface of the half round channel forms a base 105 of the profiled channel 102. The sides of the profiled channel 102 may extend outwards from the base 104 of the profiled channel 102 along the groove 117 to form a top surface 119A and 119B on opposite sides of the groove 117. The concave groove of the profiled channel 102 may be sized such that a user may place a therapeutic device into the concave groove of the channel 102 for delivery via the catheter extension device 100. The concave groove of the profiled channel 102 may receive the therapeutic device without damage to any surfaces of the therapeutic device. The catheter extension device 100 may be used to deliver therapeutic devices including but not limited to stents, coronary stents, peripheral stents, embolic coils, embolic retrieval devices, embolic spheres, embolic particles, percutaneous transluminal coronary angioplasty (PTCA) balloon, percutaneous transluminal angioplasty (PTA) balloon, and the like. The therapeutic device may be inserted through the profiled channel 102 for delivery through the distal extension segment 110.

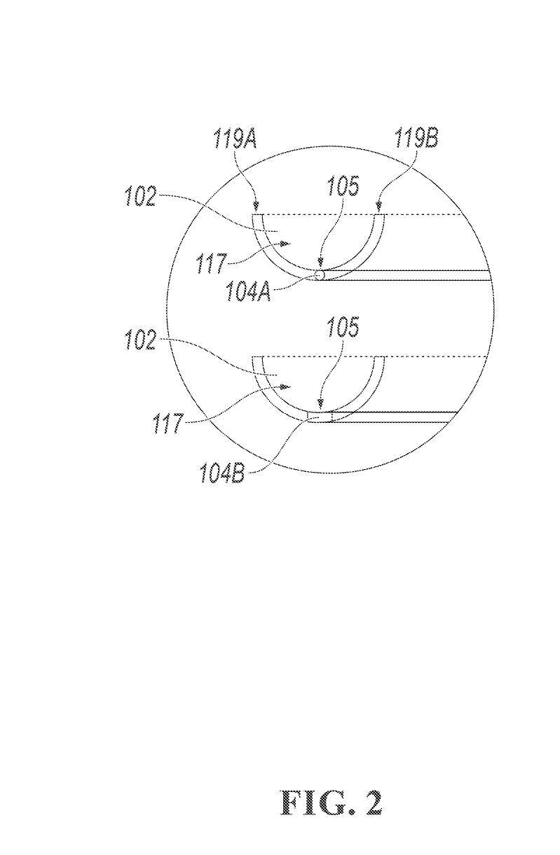

Turning to FIG. 2, the profiled channel 102 at the proximal end 101 of the catheter extension device 100 is shown. The profiled channel 102 may further comprise the channel reinforcement 104 such that the channel reinforcement 104 may be positioned within the base 105 of the profiled channel 102. The channel reinforcement 104 may provide additional support to the profiled channel 102. The channel reinforcement 104 may extend along the entire length of the profiled channel 102. The channel reinforcement 104 may also extend throughout the catheter extension device 100 including the collar 106 and the distal extension segment 110, or just the collar 106. The channel reinforcement 104 may provide stable support for tracking the catheter extension device 100 through the vasculature.

As shown in FIG. 2, in an embodiment, the channel reinforcement 104A may be formed in the shape of a round rod embedded within the base 105 of the profiled channel. Alternatively, the channel reinforcement 104b may be formed in the shape of a flat wire and similarly embedded within the base 105. Alternatively, the channel reinforcement 104 may be constructed with other shapes including but not limited to rods having cross-sectional shapes of ovals, rectangles with rounded edges, parallelograms with rounded edges, and the like. In an embodiment, the channel reinforcement 104 may comprise a guidewire coated or uncoated with Polytetrafluoroethylene (PTFE), silicone, or another lubricious agent. The channel reinforcement 104 may range from 0.010-inch diameter to 0.038-inch diameter and between 50 centimeters to 280 centimeters in length. The channel reinforcement 104 may also be made from materials including but not limited to steel, aluminum, iron, metal, metal alloys, and plastic based core rods and the like.

In one aspect, the profiled channel 102 may also be coated with a lubricious and/or anticoagulant coating. Coating the profiled channel may increase tracking and/or thrombosis prevention when coaxially passing the catheter extension device through a guiding catheter or a guiding sheath. Lubricious coatings include but are not limited to coatings exhibiting hydrophobic, hydrophilic, smooth, slippery, and or slick properties that enable the catheter extension device to be smoothly tracked through a catheter device such as the guide catheter, guiding sheath, and the like.

In an embodiment, the channel reinforcement 104 may be made of metals that may contain impurities or microscopic surface defects where thrombus is capable of being collected. The profiled channel 102 may therefore also be jacketed with polymer based material to prevent thrombus from forming around the profiled channel and/or the metal based channel reinforcement. Polymer based materials used to jacket the profiled channel may include but is not limited to pebax, nylon, polyether block amide, urethane, PTFE, and the like.

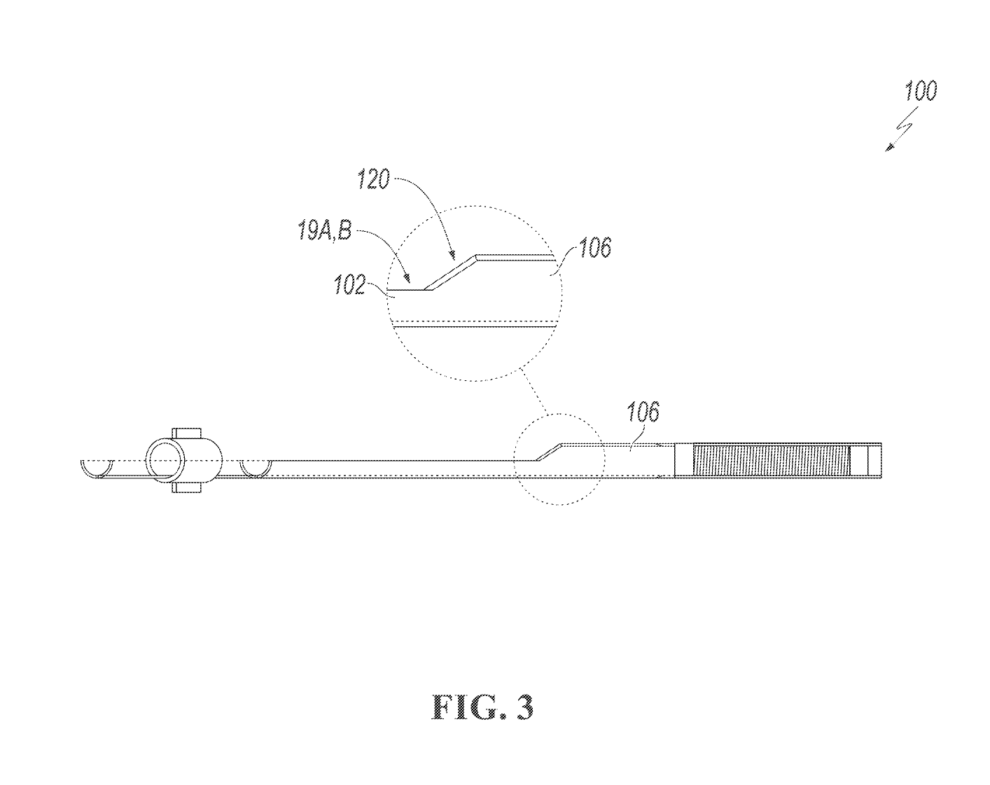

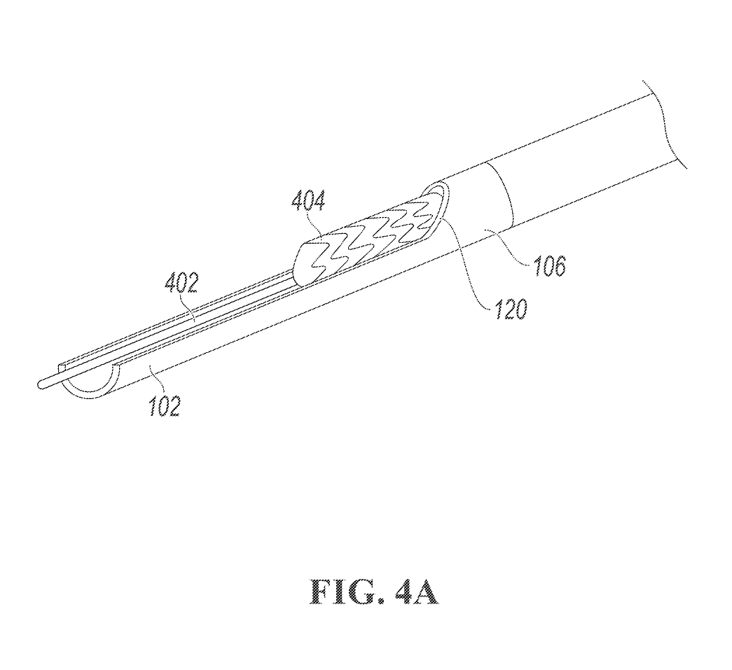

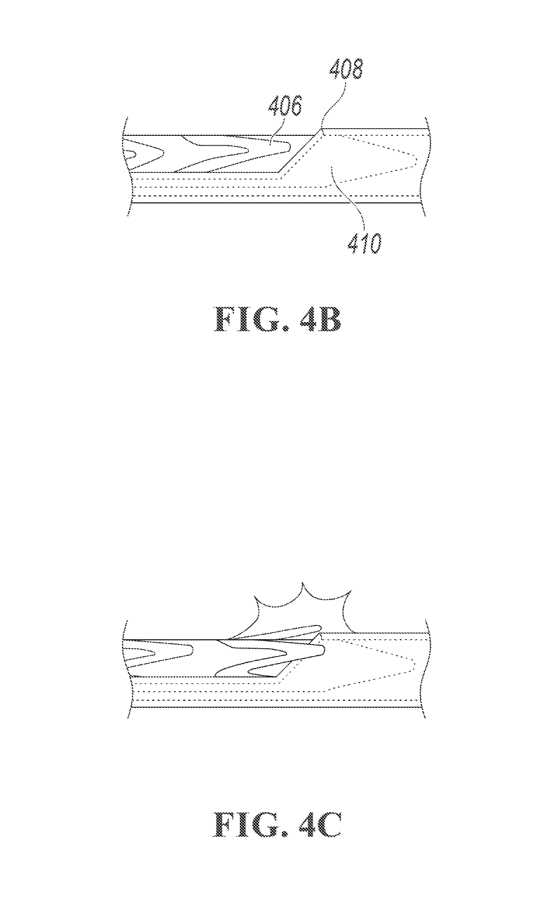

Turning to FIGS. 3 and 4A-C, the collar 106 of the catheter extension device 100 connects the profiled channel 102 to the distal extension segment 110. The portion of the profiled channel 102 contacting the collar 106 may comprise an upwards-tapered cut 120 extending from the top surface 119A and 119B of the profile channel 102 towards the cylindrical tubular portion of the collar 106. The edge 408 between the upwards-tapered cut 120 and the collar 106 contacting the inner surface of the collar 106 may be polished and rounded to prevent damage to the therapeutic device 404 being inserted into the extension device 100. FIGS. 4A-4C show a stent and deflated balloon 404 being inserted using a stent shaft 402 from the profiled channel 102 into the collar 106. As shown in FIG. 4B, the polished and rounded edge 408 may minimize the chance or prevent the struts 406 of the stent and balloon device 404 from fraying which may lead to damage of the lumen of the extension device 100 and vessel walls. FIG. 4C shows an example of the stent strut 404 frayed and damaged as the balloon tip 410 is inserted into the collar 106.

The collar 106 may be formed such that it may sustain the bond and tensile strength required between the contacting portions of the collar 106 and the distal extension segment 110 to prevent separation. The collar 106 may be formed from a single or compounded polyamide fused and shaped using heat. Other methods known to one of ordinary skill in the art may be used in forming the collar. The distal extension segment 110 may be connected to the collar 106 by being bonded or attached to the collar 106. The distal extension segment 110 may be connected by being welded, melted, fused, using an adhesive, brazing, soldering, or other fusing/joining methods known to one of ordinary skill in the art. Alternatively, the addition of the distal extension segment 110 as part of the catheter extension device 100 during manufacturing may be optional such that it may be at the discretion of the manufacturer to include the distal extension segment 110 when manufacturing the extension device 100. The distal extension segment 110 may be modular such that it is manufactured separately and may instead be later attached to the profiled channel 102 at the collar 106 prior to use. This allows for the catheter extension device 100 to be customized with various distal extension segments 110.

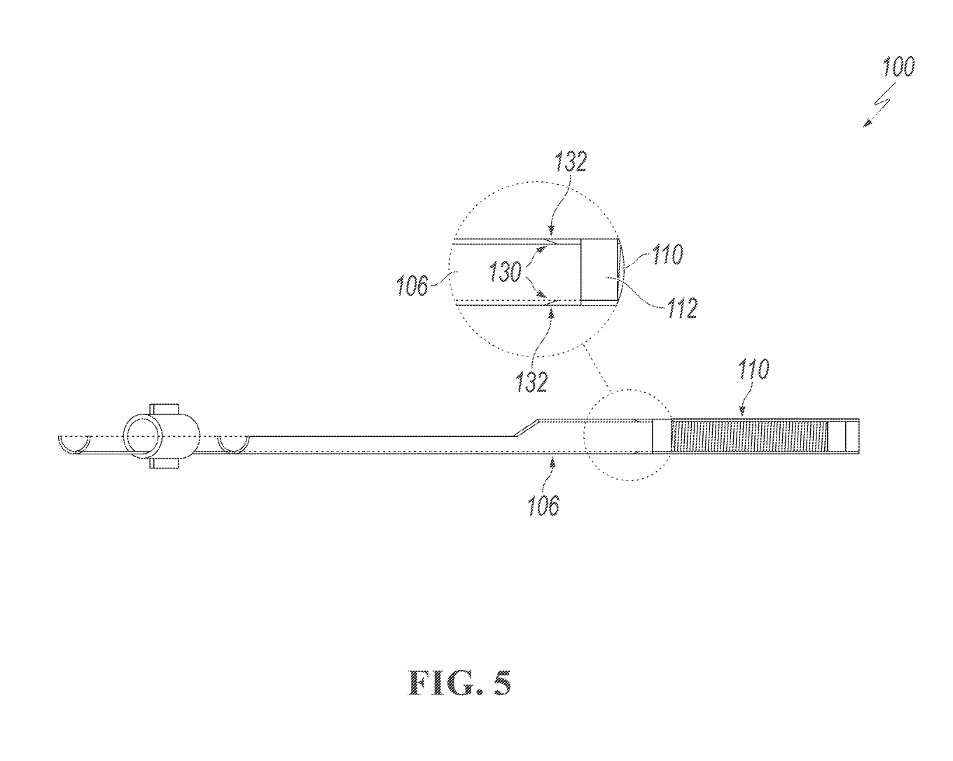

Turning to FIG. 5, in an embodiment, the contact points between the collar 106 and the distal extension segment 110 may be connected by welding the two components together. The contacting ends of the collar 106 and the distal extension segment may 110 be tapered such that the two ends may fit together temporarily stabilizing the connected ends prior to being welded together. The tapered ends may also increase the welding area and tensile strength of the connecting ends after the welding process. Each of the ends of the collar 106 and distal extension segment 110 may be correspondingly tapered to create a male and female fitting. In the example shown in FIG. 5, the end of the collar 106 may be tapered down on the outer diameter as in 130, and the end of the distal extension segment 110 may tapered down on the inner diameter as in 132. The tapering on the outer diameter of the collar 106 creates a male contacting end that fits into a female contacting end created by the tapering on the inner diameter of the distal extension segment 110. Alternatively, the end of the collar 106 may instead be tapered on the inner diameter to create a female end that fits with the male end of the distal extension segment 110 formed by being tapered on the outer diameter.

In an embodiment, the distal extension segment 110 may comprise various designs including a distal guide catheter extension segment, a distal guiding sheath extension segment, and the like. The modular distal extension segment 110 allows for customization of the catheter extension device 100 manifesting with the variety of designs possible for the distal extension segment 110. Each of the various designs for the distal extension segment 110 may be selected at the discretion of the user based the procedure for which the catheter extension device 100 is to be used with. The distal extension segments 110 may be modular such that the distal extension segments are constructed separately from the proximal profiled channel 102 allowing the distal attachment to be custom for the procedure in which its function applies. Materials used in making the variety of designs for the distal extension segment 110 include but is not limited to nylon, urethane, ethylene, and polyamides of a variety of densities and durometers. The distal extension segment 110 may also be manufactured with varying grades of stiffness, flexibility, radiopacity, and pushability.

The distal extension segment 110 may be formed comprising an inner, middle, and outer layer to provide additional reinforcement to this portion of the catheter extension device 100. The inner layer may comprise a lubricious inner liner. The middle layer may be a metallic, fiber structure, coil, or braid to provide reinforcement and prevent radial forces from collapsing the lumen. And the outer layer may be made of materials including but not limited to plastic based nylons, urethanes, polyethylene and other polyamide plastic based materials. The distal extension segment may comprise different diameters ranging from 0.035 inch to 0.184 inch depending on the application.

The profiled channel 102 may enable the facile coaxial placement of the therapeutic device proximal to the distal extension segment 110. The concave groove of the profiled channel 102 allows the catheter extension device 100 to easily receive, hold, and carry the therapeutic device to be delivered. Once inserted into the profiled channel 102, the therapeutic device may then be tracked along the channel 102 safely without exposure to metallic surfaces. The catheter extension device 100 may be used such that the therapeutic device may be coaxially placed in the profile channel at the proximal end of the catheter extension device. The therapeutic device may then be coaxially tracked through the distal extension segment 110 and staged for use in an interventional vascular procedure. As previously mentioned, the distal extension segment 110 may be modular and various designed may be used for respective procedures as pertaining to interventional radiology, interventional cardiology, interventional neurology, and other peripheral interventional procedures. The catheter extension device 100 may prevent, add, and/or sustain axial support so that the extension device 100 may be seated and stable to access selected targets.

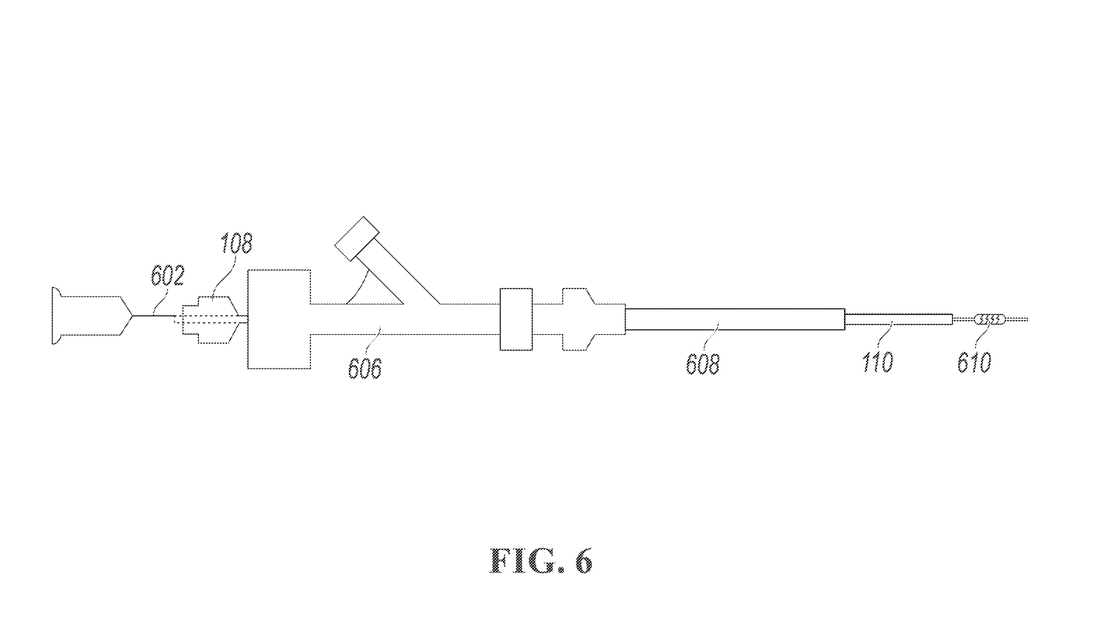

Turning to FIG. 6, an example of the catheter extension device 100 used in conjunction with a guiding catheter 608 to deliver a therapeutic device is shown. The catheter extension device 100 shown is being used to assist the guiding catheter 608 with the delivery of a stent 610. The guiding catheter 608 may be initially connected to a rotating hemostasis valve 606. After the guiding catheter 608 is seated, the catheter extension device 100 may then be inserted through the hemostasis valve 606 and tracked through the guiding catheter 608. As shown in FIG. 6, the trackable hub 108 may be used to track the catheter extension device 100. The distal extension segment 110 of the catheter extension device 100 may then further extend from the guiding catheter 608 to access selective target sites. Using the stent shaft 602, the stent 610 may then be delivered via the catheter extension device 100 to the desired target site.

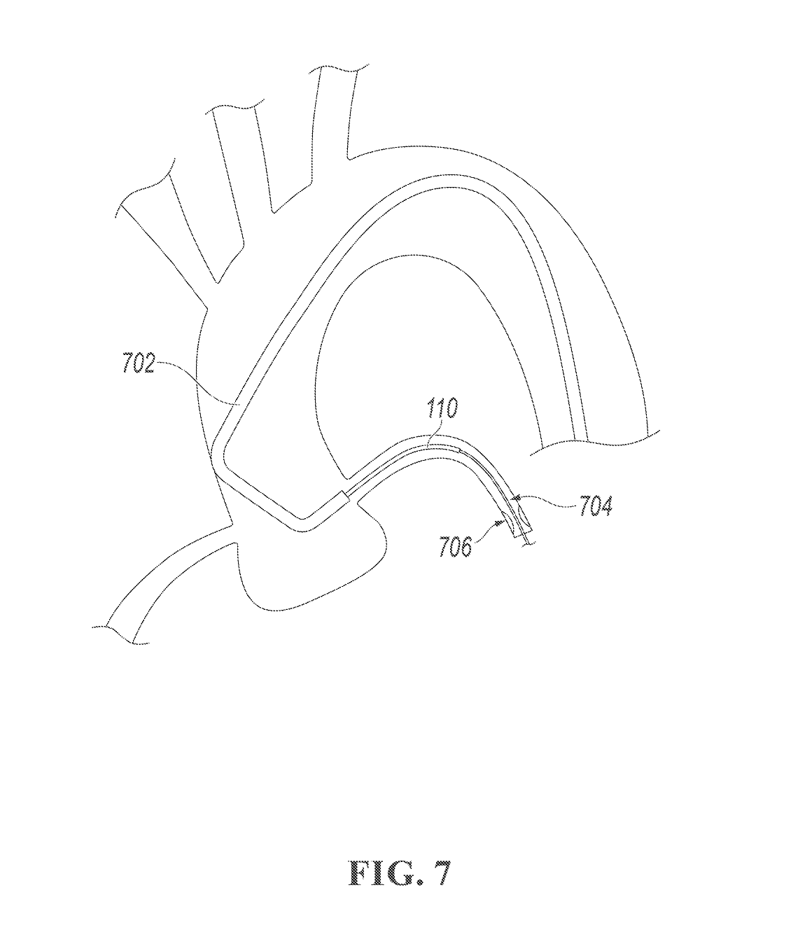

The modular distal extension segment 110 allows for the customization of the catheter extension device 100 thereby enabling the device 100 to be used in various interventional procedures. In FIG. 7, an example of the catheter extension device 100 used with a guiding catheter 702 is shown for a coronary intervention procedure. The distal extension segment 110 is shown extending from the seated guide catheter 702. The user may then further track catheter extension device 100 towards a distal lesion 706 before using a coronary guidewire 704 to cross the lesion 702.

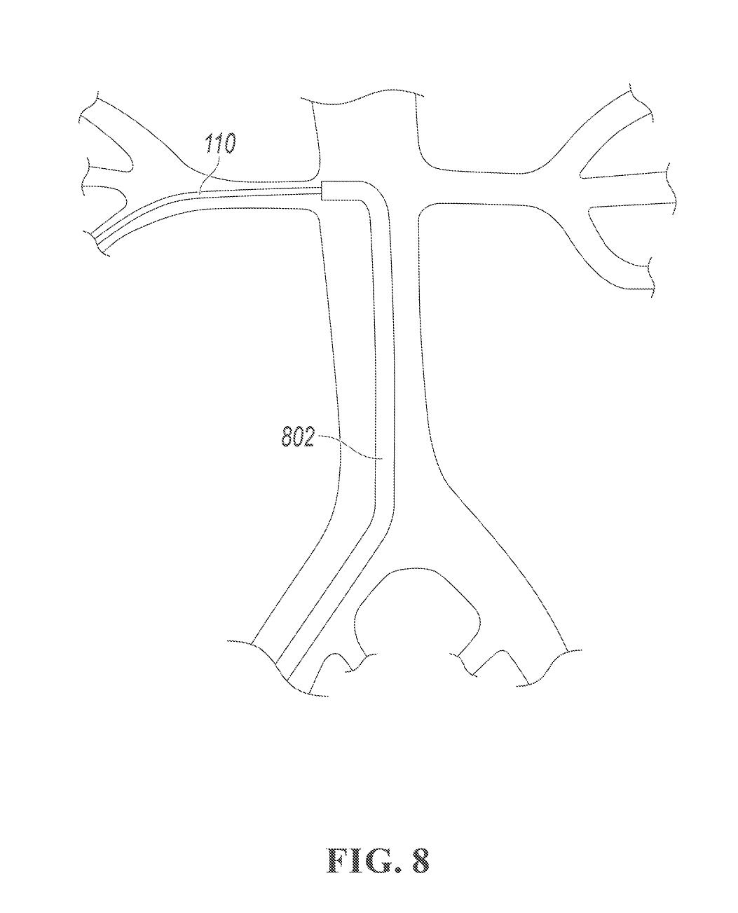

FIG. 8 shows another example of the catheter extension device 100 being used with a guiding sheath 802 for a peripheral intervention procedure. The user may track and seat the guiding sheath 802 at the ostium before using the catheter extension device 100. The user may then track the catheter extension device 100 through the guiding sheath 802 such that the distal extension segment 110 may extend from the end of the guiding sheath to extend access to vessels not previously reached by the guiding sheath 802.

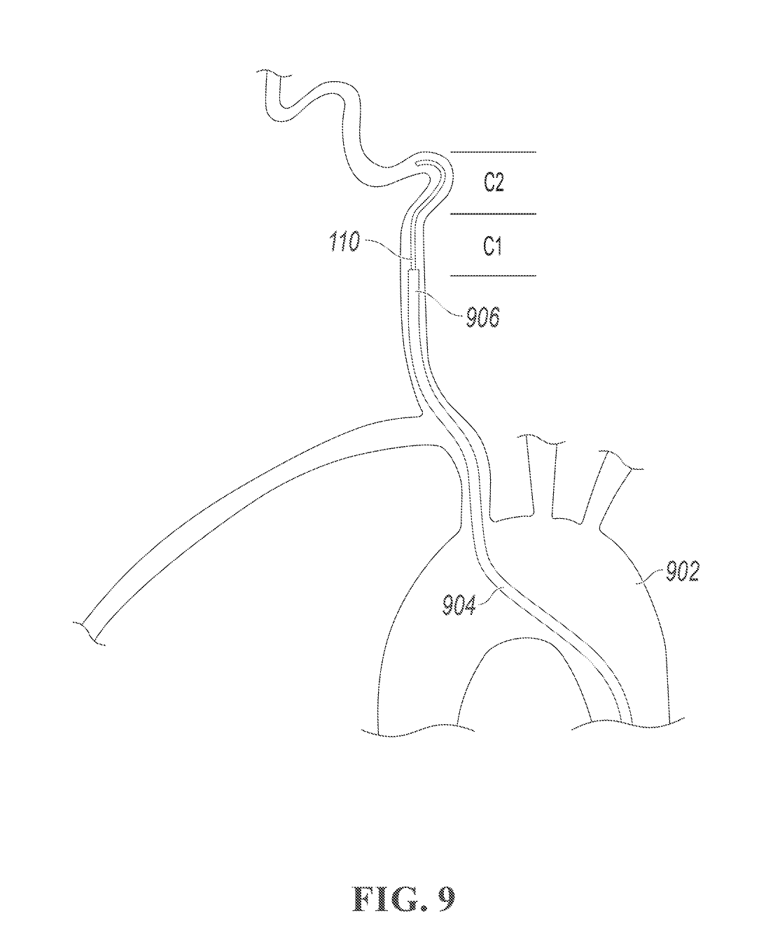

Turning to FIG. 9, in an example, the catheter extension device 100 may also be used in neurovascular applications to assist in tracking a guiding catheter 904 through the ascending aorta 902, common carotid artery, and the internal carotid artery 906. The user may typically seat the guiding catheter 904 in the opening of the cervical segment C1 of the internal carotid artery 906. The catheter extension device 100 enables the user to further access the cervical segment C1 or the petrous segment C2 for more selective and targeted delivery of therapeutic devices. In FIG. 9, the distal extension segment 110 is shown protruding from the tip of the guiding catheter 906 and accessing the cervical and petrous segments C1, C2 of the internal carotid artery 906. The combination of the standard guiding catheter 804 and the catheter extension device 100 gives the user proximal support and stability with distal access to these selective target sites.

The distal extension segments 110 may also be varied in design, material, construction, or form depending on the preference of the user and intended procedure. In an embodiment, the distal extension segment 100 may comprise a softer coil reinforced extension segment. Alternatively, a stiffer or medium stiff distal coil with braid reinforced extension segment may be used. The medium stiff embodiment of the distal extension segment 110 may be applicable for peripheral intervention in selective sites for delivery of certain therapeutic agents.

Having thus described the present invention by reference to certain of its exemplary embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of exemplary embodiments. Accordingly, it is appropriate that any claims supported by this description be construed broadly and in a manner consistent with the scope of the invention.

We claim:

1. A catheter extension device, comprising:
a device body further comprising:
a proximal end and a distal end;
a proximal segment extending from the proximal end;
a first distal segment extending from the distal end towards the proximal end; and
a tracking hub;
wherein the proximal segment further comprises a half-round tube shape; and
wherein the tracking hub is slidably engaged over the proximal segment of the device body.

2. The catheter extension device in claim 1, wherein the device body further comprises a collar segment joining the proximal segment and the first distal segment.

3. The catheter extension device in claim 1, wherein the first distal segment comprises an inner layer, a middle layer, and an outer layer, and wherein the inner layer comprises a lubricious material.

4. The catheter extension device in claim 1, wherein the first distal segment comprises an inner layer, a middle layer, and an outer layer, and wherein the middle layer is configured with a reinforced structure comprising a metallic braided fiber or a metallic coiled fiber.

5. The catheter extension device in claim 1, wherein the first distal segment comprises an inner layer, a middle layer, and an outer layer, and wherein the outer layer comprises a polyamide plastic based material.

6. The catheter extension device in claim 1, wherein the device body further comprises a support rod within the proximal segment.

7. The catheter extension device in claim 1, wherein the device body further comprises a lubricious coating comprising silicone or polytetrafluorethylene (PTFE).

8. The catheter extension device in claim 1, wherein the device body further comprises an anticoagulant coating.

9. The catheter extension device in claim 1, wherein the device body further comprises a jacketed coating comprising pebax, nylon, polyether block amide, urethane, or polytetrafluorethylene (PTFE).

10. The catheter extension device in claim 1, wherein the device body is configured to receive a therapeutic device by inserting the therapeutic device into the proximal segment.

11. The catheter extension device in claim 1, wherein the first distal segment comprises a guide catheter extension segment.

12. The catheter extension device in claim 1, wherein the first distal segment comprises a guide sheath extension segment.

13. The catheter extension device in claim 1, wherein the device body is configured to be utilized with a guiding catheter, and wherein when the device body is engaged within the guiding catheter, the device body provides coaxial stabilization to the guiding catheter.

14. The catheter extension device in claim 1, wherein the device body is configured to deliver a therapeutic device comprising of guide wires, guiding catheters, coronary stents, peripheral stents, embolic coils, embolic retrieval devices, embolic spheres, or embolic particles.

15. The catheter extension device in claim 1, wherein the device body is configured to be utilized with a guiding catheter, and wherein when the device body is engaged within the guiding catheter, the device body provides the guiding catheter with added and sustained axial support to increase stabled engagement of the guiding catheter in an arterial or vessel branch.

16. The catheter extension device in claim 1, wherein the tracking hub comprises a luer hub.

17. The catheter extension device in claim 1, wherein the tracking hub is configured to track forwards and backwards along the proximal segment.

18. The catheter extension device in claim 1, wherein the device body is configured to be utilized with a guiding catheter, and wherein the tracking hub is configured to track the device body through the guiding catheter.

19. The catheter extension device in claim 2, wherein the first distal segment is attached to a distal end of the collar segment.

20. The catheter extension device in claim 2, wherein an upwards tapered cut is formed at a transition region between the proximal segment and the collar segment, and wherein the upwards tapered cut extends from the proximal segment towards the collar segment.

21. The catheter extension device in claim 2, wherein the collar segment comprises an interior edge at a transition region between the proximal segment and the collar segment, and wherein the interior edge is rounded from an interior surface of the collar segment to an exterior surface of the collar segment.

22. The catheter extension device in claim 2, wherein the proximal segment and the collar segment comprise a unitary structure.

23. The catheter extension device in claim 2, wherein the device body further comprises a support rod within the proximal segment and the collar segment.

24. The catheter extension device in claim 2, wherein the tracking hub is configured to track forwards and backwards along the proximal segment and the collar segment.

25. The catheter extension device in claim 2, wherein the device body further comprises a lubricious coating comprising silicone or polytetrafluorethylene (PTFE).

26. The catheter extension device in claim 2, wherein the device body further comprises an anticoagulant coating.

27. The catheter extension device in claim 2, wherein the device body further comprises a jacketed coating comprising pebax, nylon, polyether block amide, urethane, or polytetrafluorethylene (PTFE).

28. The catheter extension device in claim 6, wherein the device body is configured to deliver a therapeutic device inserted into the proximal segment through the first distal segment, and wherein the therapeutic device can be tracked from the proximal segment through the first distal segment without exposing the therapeutic device to a metallic surface.

29. The catheter extension device in claim 6, wherein the support rod comprises steel, aluminum, iron, metal, metal alloys, or plastic.

30. The catheter extension device in claim 6, wherein the support rod comprises a diameter in the range from 0.010-inch to 0.038-inch.

31. The catheter extension device in claim 6, wherein the support rod comprises a length in the range from 50 centimeters to 280 centimeters.

32. The catheter extension device in claim 6, wherein the device body is configured to deliver a therapeutic device inserted into the proximal segment through the first distal segment, and wherein the therapeutic device can be tracked from the proximal segment through the first distal segment without exposing the therapeutic device to a metallic surface.

33. The catheter extension device in claim 19, wherein the first distal segment is modular and removably attached to the distal end of the collar segment, wherein the collar segment is configured to enable replacement of the first distal segment with a second distal segment differing in external diameter, stiffness, flexibility, characteristics of reinforcing layers, or location of stiffness regions, relative to the first distal segment.

34. The catheter extension device in claim 23, wherein the support rod comprises steel, aluminum, iron, metal, metal alloys, or plastic.

35. The catheter extension device in claim 23, wherein the support rod comprises a diameter in the range from 0.010-inch to 0.038-inch.

36. The catheter extension device in claim 23, wherein the support rod comprises a length in the range from 50 centimeters to 280 centimeters.

37. The catheter extension device in claim 23, wherein the device body is configured to deliver a therapeutic device inserted into the proximal segment through the first distal segment, and wherein the therapeutic device can be tracked from the proximal segment through the first distal segment without exposing the therapeutic device to a metallic surface.

38. The catheter extension device in claim 33, wherein the second distal segment is configured for use in interventional radiology, interventional cardiology, interventional neurology, or other peripheral interventional radiology procedures.
